(19) United States
(12) Patent Application Publication
(10) Pub. No.: US 2026/0110428 A1
(43) Pub. Date: Apr. 23, 2026

(54) LIGHT EMITTING DEVICE AND STERILIZER INCLUDING THE SAME

(71) Applicant: SEOUL VIOSYS CO., LTD., Ansan-si (KR)

(72) Inventors: JaeHak JEONG, Ansan-si (KR); KiYon PARK, Ansan-si (KR)

(21) Appl. No.: 19/348,933

(22) Filed: Oct. 3, 2025

Related U.S. Application Data
(60) Provisional application No. 63/708,727, filed on Oct. 17, 2024.
(60) Provisional application No. 63/739,173, filed on Dec. 27, 2024.

(51) Int. Cl. F21V 33/00; A61L 2/10; F21Y 115/10

(52) U.S. Cl. CPC F21V 33/0064; A61L 2/10; A61L 2202/11; F21Y 2115/10

(57) ABSTRACT

A light emitting device including: a device substrate; a device housing disposed on the substrate and including a cavity exposing at least a region of the substrate to an outside; a light emitter disposed in the region of the substrate and having a light-emitting surface configured to generate light; and an optical element disposed above the device housing and configured to refract light from the light emitter, in which a surface of the optical element has a first curvature in a region that intersects a virtual line extending from a center of the light emitter at a first angle with respect to the light-emitting surface, and a second curvature that is different from the first curvature in a region that intersects a virtual line extending from the center of the light emitter at a different, second angle with respect to the light-emitting surface.

### CROSS REFERENCE TO RELATED APPLICATIONS

This application claims priority from and the benefit of U.S. Provisional Patent Application No. 63/708,727 , filed on Oct. 17, 2024, and United States Provisional Patent Application No. 63/739,173 , filed on Dec. 27, 2024, each of which is hereby incorporated by reference for all purposes as if fully set forth herein.

### BACKGROUND

### Field

Embodiments of the invention relate generally to a light emitting device and a sterilizer including the same.

### Discussion of the Background

Recently, as viruses or fungi that poses a threat to the human body have become increasingly prevalent, efforts have been made to sterilize such microorganisms in order to protect the human body from infection.

In particular, electronic products that have a sterilization function capable of sterilizing home appliances associated with food, such as water purifiers or refrigerators, are gaining attention. Furthermore, there is a growing demand for a light source that sterilizes regions having various shapes in order to provide an effective sterilization function.

The above information disclosed in this Background section is only for understanding of the background of the inventive concepts, and, therefore, it may contain information that does not constitute prior art.

### SUMMARY

According to embodiments of the invention, a light emitting device and a sterilizer including the same are capable of sterilization.

A light emitting device and a sterilizer including the same according to embodiments of the invention are capable of uniformly irradiating light to a sterilization region.

A light emitting device and a sterilizer including the same according to embodiments of the invention are capable of reducing degradation of sterilization power caused by a distance difference between the light emitting device and a sterilization region.

A light emitting device and a sterilizer including the same according to embodiments of the invention are capable of automatically performing sterilization.

Additional features of the inventive concepts will be set forth in the description which follows, and in part will be apparent from the description, or may be learned by practice of the inventive concepts.

A light emitting device according to an embodiment includes: a device substrate; a device housing disposed on the substrate and providing a cavity for exposing a region of the substrate to an outside; a light emitter disposed in the region of the substrate and having a light-emitting surface configured to generate light; and an optical element disposed above the device housing so that the light from the light emitter is refracted, wherein a surface of the optical element has a first curvature in a region that intersects a virtual line extending from a center of the light emitter at a first angle with respect to the light-emitting surface, and has a second curvature, different from the first curvature, in a region that intersects a virtual line extending from the center of the light emitter at a second angle, different from the first angle, with respect to the light-emitting surface.

A center region of the surface of the optical element, located directly above the light emitter, may have the largest curvature.

A center of curvature of the center region may be disposed above the light emitter.

An edge region of the surface of the optical element, adjacent to the device housing, may have the smallest curvature.

The optical element may refract light to form a light distribution curve with an illuminance of 50% or more within a radiant intensity distribution angle ranging from −45°to 45°.

A plurality of main peaks may be formed in the light distribution curve.

The plurality of main peaks may include: a first main peak formed at the radiant intensity distribution angle less than 0°; and a second main peak formed at the radiant intensity distribution angle greater than 0°.

Normalized intensities of the first main peak and the second main peak may be different from each other.

A lowest normalized intensity in a region between the first main peak and the second main peak may be at least 0.8 times the higher of the normalized intensities of the first main peak and the second main peak.

The first main peak and the second main peak may be formed at the radiant intensity distribution angle ranging from −20° to 20° in the light distribution curve.

A single main peak may be formed within the radiant intensity distribution angle ranging from −20° to 20°in the light distribution curve.

A plurality of sub-peaks may be formed in the light distribution curve, having a smaller normalized intensity than the plurality of main peaks and formed at a larger radiant intensity distribution angle than the main peaks, and the plurality of sub-peaks may be formed at the radiant intensity distribution angle ranging from −20° to 20°.

Normalized intensities of the plurality of sub-peaks may be at least 0.4 times the higher of the normalized intensities of the first main peak and the second main peak.

A light emitting device according to another embodiment includes: a substrate; a housing disposed on the substrate and providing a cavity for exposing a region of the substrate to an outside; a light emitter disposed in the region of the substrate and configured to generate light; an optical element supported on an upper side of the housing so that the light from the light emitter is refracted; and a plurality of sidewalls extending upward from an upper side of the housing, wherein the optical element is disposed between the plurality of sidewalls.

A sterilizer according to still another embodiment includes: a housing assembly that provides a first region and a second region; a first light emitting device configured to generate light having a first light distribution curve to sterilize the first region; and a second light emitting device configured to generate light having a second light distribution curve to sterilize the second region. The first light distribution curve and the second light distribution curve have main peaks at different radiant intensity distribution angles.

The first region may be larger than the second region.

The first light emitting device may provide a wider radiation angle than the second light emitting device.

A plurality of main peaks may be formed in the first light distribution curve, and the plurality of main peaks may be formed within the radiant intensity distribution angle ranging from −30° to 20°.

A normalized intensity of the first light distribution curve at the radiant intensity distribution angle of −30° to 20° may be at least 0.8 times the higher of normalized intensities of the first main peak and the second main peak.

A single main peak may be formed in the second light distribution curve.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic diagram illustrating a sterilizer according to a first embodiment of the invention.

FIG. 2 is a schematic isometric view illustrating a sterilization module that sterilizes a first region of the sterilizer of FIG. 1.

FIG. 3 is a schematic diagram illustrating a sterilization module of the sterilizer of FIG. 1.

FIG. 4 is a schematic diagram illustrating a light emitting device included in the sterilization module of FIG. 3.

FIG. 5 is a diagram illustrating a light distribution curve of a light emitter of the light emitting device of FIG. 1.

FIG. 6 is a diagram illustrating a first type light distribution curve of light generated from the light emitting device of FIG. 4.

FIG. 7 is a diagram illustrating a second type light distribution curve of light generated from the light emitting device of FIG. 4.

FIG. 8 is a diagram illustrating a third type light distribution curve of light generated from the light emitting device of FIG. 4.

FIG. 9 is a diagram illustrating a fourth type light distribution curve of light generated from the light emitting device of FIG. 4.

FIG. 10 is a diagram illustrating a fifth type light distribution curve of light generated from the light emitting device of FIG. 4.

FIG. 11 is a diagram illustrating a sixth type light distribution curve of light generated from the light emitting device of FIG. 4.

FIG. 12 is a diagram illustrating a light emitting device of a sterilizer according to a second embodiment of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z—axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Hereinafter, a light emitting device 100 and a sterilizer 1 including the same according to a first embodiment of the invention will be described.

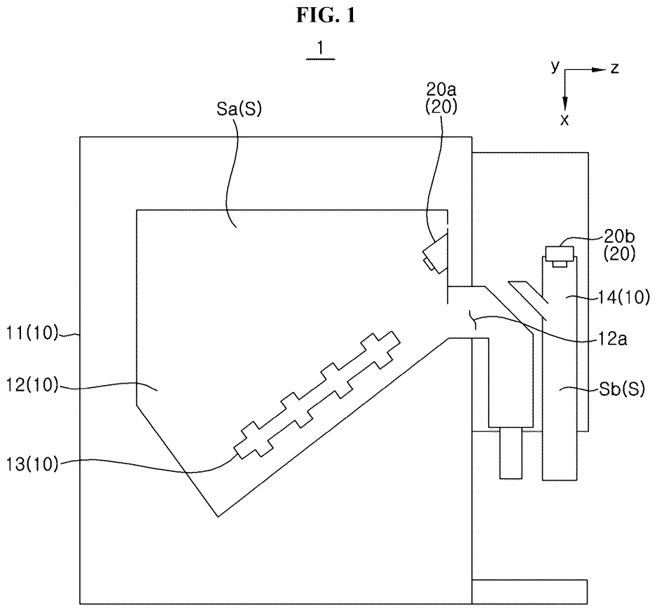

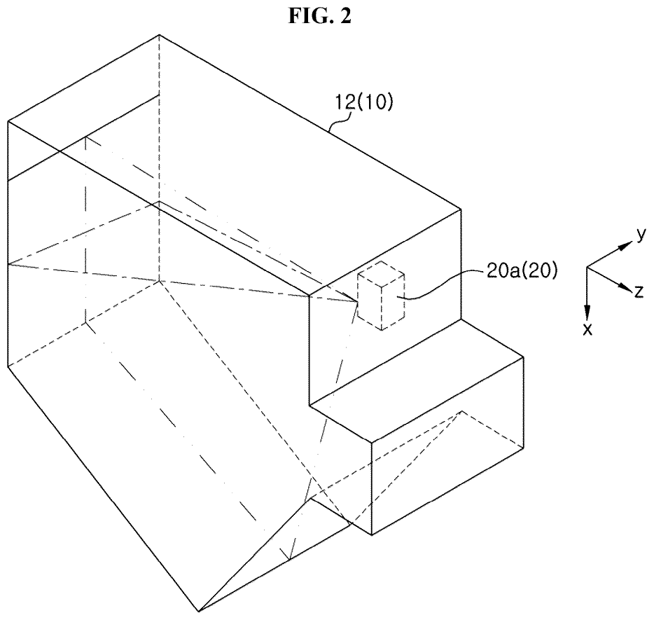

Referring to FIGS. 1 to 3, the sterilizer 1 may be an apparatus for sterilizing bacteria, foreign substances, and the like. The sterilizer 1 may be implemented as an ice water purifier, but is not limited thereto. The sterilizer 1 may provide an accommodation space for accommodating water and ice, and may sterilize the water, the ice, and the accommodation space. The sterilizer 1 may include a housing assembly 10 and a sterilization module 20.

The housing assembly 10 may support the sterilization module 20, and provide an exterior of the sterilizer 1. The housing assembly 10 may include a sterilization region S to be sterilized by the sterilization module 20. Furthermore, a plurality of sterilization regions S may be formed. The plurality of sterilization regions S may include a first region Sa and a second region Sb. The first region Sa may be formed larger than the second region Sb, and may be configured as a tank 12 to be described later. The second region Sb may be a flow channel 14 to be described later, but is not limited thereto. The first region Sa and the second region Sb may be sterilized by the sterilization module 20. The housing assembly 10 may include a frame 11, the tank 12, a conveyor 13, and the flow channel 14.

The frame 11 may form an exterior of the sterilizer 1, and may provide a space for accommodating a water purification filter, an ice making device, and the like. Furthermore, the frame 11 may support the tank 12, the conveyor 13, and the flow channel 14. In particular, water that has entered the interior of the frame 11 may be filtered into purified water by the water purification filter and then flow to the flow channel 14 or be formed into ice by the ice making device.

The tank 12 may accommodate ice. An inner surface of the tank 12 may be the first region Sa described above. Furthermore, a length of the tank 12 in a vertical direction (x-axis direction) may be formed to be longer than a length thereof in a horizontal direction (y-axis direction). In particular, the inner surface of the tank 12 may be sterilized by the sterilization module 20. The tank 12 may be supported by the frame 11 so as to be disposed below the ice making device and may accommodate ice detached from the ice making device. An outlet 12a through which ice may be discharged may be formed in the tank 12. The outlet 12a may be disposed on a front side of the tank 12. Furthermore, a bottom surface of the tank 12 may be inclined downwardly in a direction away from the outlet 12a. The inclination of the bottom surface of the tank 12 may prevent ice from accumulating near the outlet 12a. The sterilization module 20 may be disposed on one surface of the tank 12. A light exit surface of the sterilization module 20 may be inclined at a certain angle from the one surface of the tank 12. An angle formed by the light exit surface of the sterilization module 20 and the one surface of the tank 12 may be 25 degrees or more and 50 degrees or less. This allows light to be intensively delivered or concentrated to a specific region without being unnecessarily emitted in regions where sterilization is not required, thereby improving efficiency.

The conveyor 13 is disposed inside the tank 12 and may transport ice to the outlet 12a. By the conveyor 13, the ice may be discharged to the outside of the housing assembly 10 and provided to a user. The conveyor 13 may be supported by the tank 12 so as to be rotatable inside the tank 12. For example, the conveyor 13 may have a screw shape. The conveyor 13 may be sterilized together when the sterilization module 20 sterilizes the inner surface of the tank 12.

The flow channel 14 may provide a passage through which water flows. For example, the flow channel 14 may be a flow path of a faucet for discharging purified water, but is not limited thereto. The flow channel 14 may be the second region Sb described above. In particulars, the flow channel 14 may be sterilized by the sterilization module 20. The sterilization module 20 may be disposed on one surface of the flow channel 14. A light exit surface of the sterilization module 20 may be disposed substantially perpendicularly to the one surface of the flow channel 14. At this time, an angle formed by the light exit surface of the sterilization module 20 and the one surface of the flow channel 14 may be 85 degrees or more and 95 degrees or less. The light exit surface of the sterilization module 20 may be disposed substantially perpendicular to a direction in which water flows in the flow channel 14. Alternatively, the light exit surface of the sterilization module 20 may be disposed substantially parallel to the direction in which water flows in the flow channel 14. Through this, light may be intensively delivered or concentrated to a specific region without being unnecessarily emitted in regions where sterilization is not required, thereby increasing efficiency.

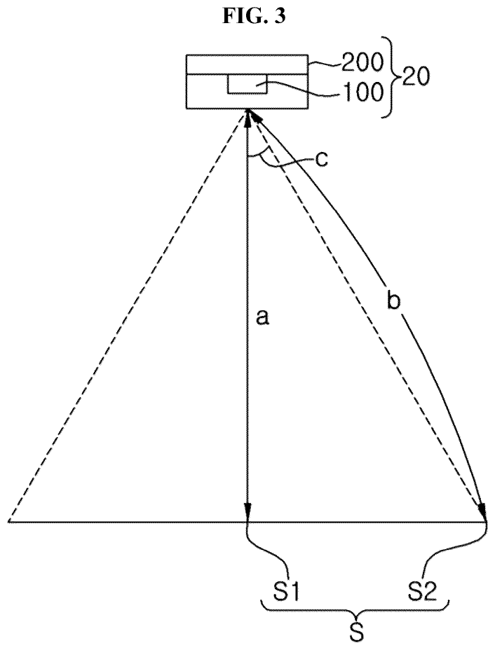

The sterilization module 20 may perform sterilization by generating light. In particular, the sterilization module 20 may sterilize the housing assembly 10. For example, light to be emitted from the sterilization module 20 may be emitted toward the sterilization region S of the housing assembly 10 to sterilize the sterilization region S. Furthermore, the sterilization region S of the housing assembly 10 to be sterilized by the sterilization module 20 may be disposed to be spaced apart from the sterilization module 20 by a predetermined distance. The sterilization region S may also be referred to as a sterilization target surface. Furthermore, the sterilization region S may include a first target region S1 and a second target region S2, as shown in FIG. 3.

The first target region S1 may be a region disposed most proximately to the sterilization module 20 within the sterilization region S. In particular, the first target region S1 may be a center region of the sterilization region S. More particularly, the first target region S1 may be a region closest to the sterilization module 20 among the sterilization target surface. The first target region S1 may be disposed to be spaced apart from the sterilization module 20 by a first distance (a). Furthermore, the first distance (a) may be a distance extending in a direction perpendicular to a light exit surface of the sterilization module 20. The first distance (a) may be about 228 mm, but is not limited thereto. In some embodiments, the first distance (a) may vary depending on a shape of the housing assembly 10 or the sterilization region S. The first distance (a) may be about 0.5 to 0.89 times a second distance (b) to be described later.

The second target region S2 may be a region disposed farthest from the sterilization module 20 within the sterilization region S. The second target region S2 may be an edge region of the sterilization region S. The second target region S2 may be disposed to be spaced apart from the sterilization module 20 by a second distance (b). The second distance (b) and the first distance (a) may form a target angle (c). The target angle (c) may be 30° or more and 60° or less. For example, the target angle (c) may be 39°.

The sterilization region S may have a preset length based on a region horizontal to the sterilization module 20. The length of the sterilization region S may be about 370 mm, but is not limited thereto. In some embodiments, the length of the sterilization region S may be changed according to the shape of the housing assembly 10 or the sterilization region S. Furthermore, when light from the sterilization module 20 is emitted to the first target region S1 and the second target region S2, the sterilization region S may be positioned so that the first target region S1 and the second target region S2 have uniform illuminance. A difference in illuminance between the first target region S1 and the second target region S2 may be less than 50%. In particular, the sterilization region S may be uniformly sterilized.

The sterilization modules 20 may be formed in plural. The plurality of sterilization modules 20 may have different light distribution characteristics. For example, one of the plurality of sterilization modules 20 may have a wide light distribution characteristic to sterilize the sterilization region S having a relatively large region. Furthermore, another one of the plurality of sterilization modules 20 may have a relatively narrow light distribution characteristic to sterilize the sterilization region S having a relatively narrow region. The plurality of sterilization modules 20 may be disposed in different regions. Light exit directions of the plurality of sterilization modules 20 may be formed differently. This helps minimize optical interference between the plurality of sterilization modules 20 and reduce accumulation of optical damage, thereby increasing reliability. The plurality of sterilization modules 20 may include a first sterilization module 20a and a second sterilization module 20b.

The first sterilization module 20a may be disposed at a different position from the second sterilization module 20b to sterilize the first region Sa of the housing assembly 10. In particular, the first sterilization module 20a may generate light toward the inner surface of the tank 12 to sterilize the inner surface of the tank 12. Furthermore, the first sterilization module 20a may also sterilize the ice and the conveyor 13 while sterilizing the inner surface of the tank 12. The first sterilization module 20a may be disposed above the tank 12. Furthermore, the first sterilization module 20a may emit light in a direction away from the second sterilization module 20b. The first sterilization module 20a may have a wider light distribution characteristic than the second sterilization module 20b to sterilize the first region Sa having a relatively large region.

The first light source module 100-1 may irradiate the first region Sa with light for a certain period of time, so the illuminance per unit region applied to the first region Sa may be lower than the illuminance per unit region applied to the second region Sb.

The second sterilization module 20b may sterilize the second region Sb of the housing assembly 10. For example, the second region Sb may be implemented as a drain pipe, a cock, or the flow channel 14. In particular, the second sterilization module 20b may generate light toward the flow channel 14 to sterilize the flow channel 14. The second sterilization module 20b may be disposed above the flow channel 14 to generate light downward. The second sterilization module 20b may have a relatively narrow light distribution characteristic to sterilize the second region Sb having a relatively narrow region. The second sterilization module 20b may provide a higher illuminance to the second region Sb than the first sterilization module 20a.

The sterilization module 20 may include a light emitting device 100 and a supporter 200.

The light emitting device 100 may generate light. In particular, the light emitting device 100 may generate light for sterilizing the housing assembly 10. The light emitting device 100 may be formed to extend in one direction. More particularly, a length of the light emitting device 100 in one direction may be formed to be longer than a length thereof in another direction perpendicular to the one direction. Hereinafter, the length of the light emitting device 100 in one direction is referred to as a long axis, and the length in the other direction is referred to as a short axis. Furthermore, the light emitting device 100 included in the first sterilization module 20a is referred to as a first light emitting device, and the light emitting device 100 included in the second sterilization module 20b is referred to as a second light emitting device.

The first light emitting device and the second light emitting device may have different light distribution characteristics. The first light emitting device may generate light for sterilizing a wide sterilization region S, and the second light emitting device may generate light for sterilizing a narrow sterilization region S.

The first light emitting device of the first sterilization module 20a may be disposed in the first region Sa such that its length in one direction is disposed in the vertical direction (x-axis direction) and its length in the other direction is disposed in the horizontal direction (y-axis direction). Furthermore, the first light emitting device of the first sterilization module 20a may irradiate the first region Sa with light for a certain period of time. Furthermore, an illuminance of the first light emitting device of the first sterilization module 20a per unit region may be lower than an illuminance of the first light emitting device of the second sterilization module 20b. Furthermore, the first light emitting device of the first sterilization module 20a may emit light with a larger radiation angle than the second light emitting device of the second sterilization module 20b.

Furthermore, the second light emitting device of the second sterilization module 20b may irradiate the second region Sb with light for a shorter time than the first sterilization module 20a. An illuminance of the second light emitting device of the second sterilization module 20b per unit region may be higher than an illuminance of the first light emitting device of the first sterilization module 20a.

The sterilization module 20 may not include a wavelength conversion member, but the inventive concepts are not limited thereto. In some embodiments, the light emitting device 100 may further include a wavelength conversion member. Furthermore, at least one of the plurality of sterilization modules 20 may further include a wavelength conversion member, or may include a wavelength conversion material dispersed in an encapsulant or a lens, in other embodiments.

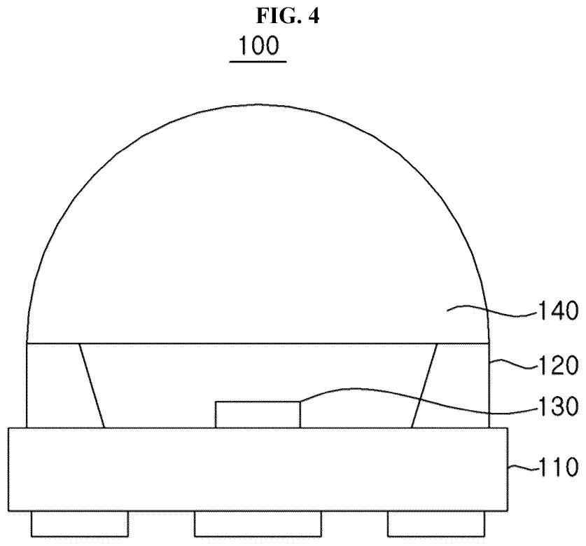

Referring further to FIG. 4, each light emitting device 100 may include a device substrate 110, a device housing 120, a light emitter 130, and an optical element 140.

The device substrate 110 may support the device housing 120, the light emitter 130, and the optical element 140. Hereinafter, the device substrate 110 is referred to as a substrate. For example, the substrate 110 may be a printed circuit board (PCB). Furthermore, the substrate 110 may include one or more of Cu, Zn, Au, Ni, Al, Mg, Cd, Be, W, Mo, Si, Ag, and Fe, or an alloy thereof. However, the inventive concepts are not limited thereto, and the substrate 110 may include one or more of FR1, CEM-1, and FR-4 in some embodiments. Here, FR1 is a material in which a copper foil and a laminate paper are stacked, and CEM-1 is a material in which a copper foil, a glass fiber fabric, a laminate paper, and a glass fiber fabric are sequentially stacked. Furthermore, FR-4 is a material in which a copper foil and a glass fiber fabric or a glass fiber fabric are stacked. In addition, the substrate 110 may include a layer made of an insulating material such as a ceramic like alumina (Al₂O₃), aluminum nitride (AlN), or zirconia toughened alumina (ZTA), or a polymer compound such as epoxy resin, polyphthalamide (PPA), polymethyl methacrylate (PMMA), liquid crystal polymer (LCP), polycarbonate (PC), PBT, PET, or silicone.

The device housing 120 may be disposed in a region on the substrate 110. The device housing 120 may include a cavity formed to a predetermined depth from the upper surface downward so that a region of the substrate 110 is exposed to the outside. The depth of the cavity may be greater than the height of the light emitter 130. Furthermore, an inner wall of the device housing 120 forming the cavity may include an inclined surface to reflect light emitted from the light emitter 130 in an upward direction. A molding layer may be provided inside the device housing 120. Hereinafter, the device housing 120 is referred to as a housing. The housing 120 may be formed of various insulating materials. For example, the housing 120 may include a polymer resin such as a ceramic resin, an epoxy resin, a silicone resin, a polyimide resin, or a urethane resin. Furthermore, the housing 120 may include a reflective material for reflecting or scattering light. For example, the reflective material may include titanium oxide (TiO₂), silicon oxide (SiO₂), barium sulfide (BaS), barium sulfate (BaSO₄), or zirconium oxide (ZrO₂). In addition, the housing 120 may include a metal material for reflecting light. For example, the housing 120 may include a metal material, such as Ag, Al, Au, or Cu.

The housing 120 and the cavity may be disposed in a region on the substrate 110. The substrate 110 may include wiring for electrical connection. The cavity of the housing 120 may expose a region of the substrate 110. In the region of the substrate 110 exposed by the cavity, the light emitter 130 is mounted, and may generate and emit light by receiving power through the wiring of the substrate 110.

The light emitter 130 is supported by the substrate 110 and may generate light. The light emitter 130 is electrically connected to an electric circuit of the substrate 110, and may generate light by receiving electricity from the outside through the electric circuit. The light emitter 130 may emit light to sterilize the sterilization region S. Light emitted from the light emitter 130 may have any wavelength range that has a sterilization function. In an embodiment, the light emitter 130 may include a light-emitting diode formed of a compound semiconductor. Furthermore, the light-emitting diode applied to the light emitter 130 may be any one of a vertical structure, a horizontal structure, and a flip-chip structure. For example, the light emitter 130 may be an element that converts electrical energy into light, such as a light-emitting diode chip including a light-emitting diode, a laser diode, or an organic light-emitting diode. The light emitter 130 may generate UVC (200 nm-280 nm), UVB (280 nm-315 nm), UVA (315 nm-420 nm), blue light, green light, yellow light, red light, infrared light, and the like. Furthermore, the light emitter 130 may be composed of any one of a flip chip, a lateral chip, or a vertical chip. A light-emitting surface of the light emitter 130 may be a surface opposite to the surface disposed on the substrate 110. In this manner, light loss due to the substrate 110 may be reduced. The light emitter 130 may have a radiation angle of 120 degrees or more. The emission pattern of the light emitter may have a normalized intensity of 0.8 or more even at a radiation angle of −45 degrees or less or 45 degrees or more. An optical element 140 may be additionally disposed above the light emitter to adjust a radiation angle. This allows light to be delivered intensively or concentrated to a specific region without being unnecessarily emitted in undesired regions.

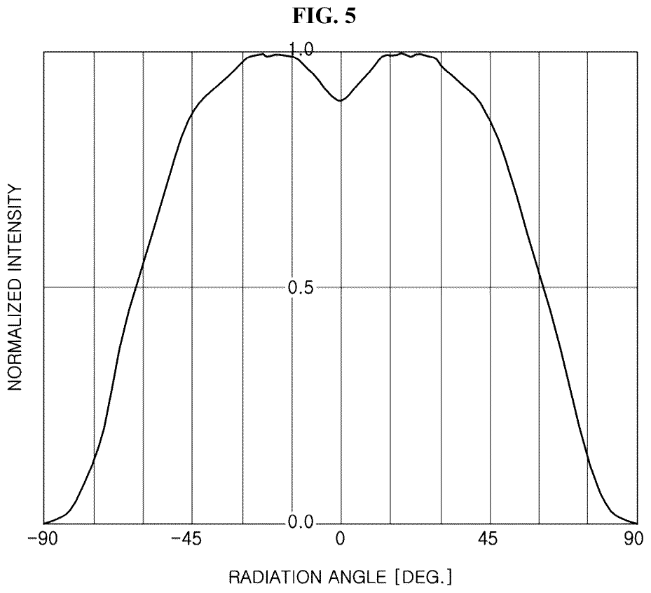

Referring to FIG. 5, the radiation angle of the light emitter 130 may be measured by using a goniophotometer. For radiation angle measurement, the light emitter 130 may be placed on a PCB or a flat substrate for measurement. The radiation angle of the light emitter 130 or the light emitting device 100 may be measured with the front of the light emitter 130 facing forward and at a position spaced 1 m away from the light emitter 130. Furthermore, the radiation angle may be an angle between a point having 50% luminous intensity between −90° and a reference line (0°), and a point having 50% luminous intensity between the reference line (0°) and +90°, by measuring the luminous intensity for each angle while rotating in a range of −90°to +90° with respect to a virtual reference line perpendicular to the light-emitting surface of the light emitter 130, compared to the largest luminous intensity.

The optical element 140 may be supported on an upper side of the housing 120 so as to be disposed above the light emitter 130. In particular, the optical element 140 may be attached to one surface of the housing 120 via an adhesive. The optical element 140 may adjust the beam angle by refracting light emitted from the light emitter 130. The optical element 140 may have a different curvature for each region. In particular, the optical element 140 may be formed to have two different curvatures according to the angle it forms with the light-emitting surface of the light emitter 130. The optical element 140 may have a smaller curvature as the angle formed with the light-emitting surface of the light emitter 130 becomes smaller. More particularly, the surface of the optical element 140 has a first curvature in a region that intersects a virtual line extending from the center of the light emitter 130 at a first angle with respect to the light-emitting surface, and may have a second curvature different from the first curvature in a region that intersects a virtual line extending from the center of the light emitter 130 at a second angle different from the first angle with respect to the light-emitting surface.

The optical element 140 may be formed such that a region perpendicular to the light-emitting surface of the light emitter 130 has the largest curvature. The optical element 140 may be formed to have a smaller curvature as the angle formed with the light-emitting surface of the light emitter 130 becomes smaller.

In particular, a center region of the surface of the optical element 140, disposed directly above the light emitter 130, may be formed to have the largest curvature. A center of curvature of the center region may be disposed above the light emitter 130. Furthermore, an edge region of the surface of the optical element 140, adjacent to the housing 120, may be formed to have the smallest curvature. Such an optical element 140 may refract light generated to the side of the light emitter 130 to form a narrow beam angle. Through this, light may be delivered intensively or concentrated to a specific region without being unnecessarily emitted in undesired regions.

A difference in orientation angle between the optical element 140 and the light emitter 130 may be 60° or more. This difference may correspond to a difference between the orientation angle of the light emitting device 100 with the optical element 140 installed and the orientation angle of the light emitting device measured with the optical element 140 removed.

The optical element 140 may be formed of a transparent material through which light passes, such as a silicone resin, an epoxy resin, glass, urethane, methyl methacrylate (M. M. A), polystyrene, allyl diglycol carbonate resin, polycarbonate resin, poly-methylmethacrylate (P. M. M. A), or Teflon resin.

The supporter 200 may support the light emitting device 100. For example, the light emitting device 100 may be disposed in a region of the supporter 200. The supporter 200 may include a power connection unit for supplying power to the light emitting device 100. Furthermore, the supporter 200 may include a controller that controls the operation of the light emitting device 100.

The supporter 200 may further include a configuration for protecting the light emitting device 100 from an external environment. For example, the supporter 200 may include a sealing part to prevent moisture penetration. In particular, the sealing part may prevent damage to the light emitting device 100 due to moisture and increase reliability.

Furthermore, the supporter 200 may further include a reflection part that reflects light toward the sterilization region S by adjusting the path of light from the light emitting device 100, an optics part that concentrates light, and the like. By the supporter 200, the sterilization module 20 may emit uniform light to the sterilization region S.

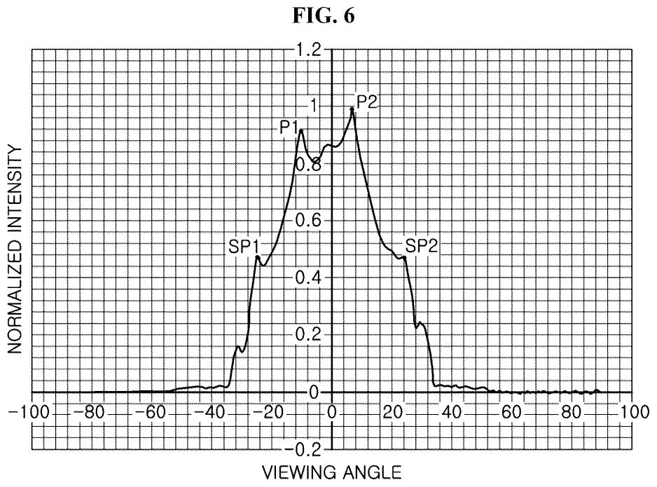

Hereinafter, the light distribution curve of the light emitting device 100 will be decribed with further reference to FIGS. 6 to 11.

The optical element 140 may be configured so that the light emitting device 100 has an orientation angle of −45° to 45°. In particular, the optical element 140 may form a light distribution curve in which light emitted from the light emitting device 100 has an illuminance (intensity) of 50% or more in the range of −45° to 45° with respect to a virtual line perpendicular to the light-emitting surface. Furthermore, a plurality of light distribution curves may be formed from the light of the light emitting device 100.

One of such plurality of light distribution curves may represent the luminous intensity of light distributed along the long axis direction of the light emitting device 100. In particular, one of the plurality of light distribution curves may be a light distribution curve that provides an orientation angle on a first virtual plane formed by the virtual line and the long axis direction of the light emitting device 100. Furthermore, another one of the plurality of light distribution curves may represent the luminous intensity of light distributed in the short axis direction (y-axis direction) of the light emitting device 100. In particular, another one of the plurality of light distribution curves may be a light distribution curve that provides an orientation angle on a second virtual plane formed by the virtual line and the short axis direction of the light emitting device 100.

Furthermore, light may be refracted by the optical element 140 so that the light emitted from the light emitting device 100 has a radiation angle of −45° to 45° as described above.

Furthermore, the light distribution curve of light generated from the light emitting device 100 may be formed to have one or more main peaks. Meanwhile, the light distribution curve may be formed by the light emitting device 100 for irradiating the sterilization region S with constant light.

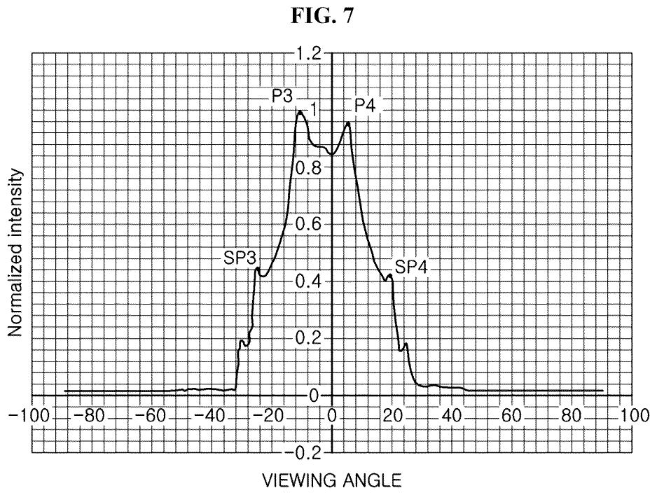

Referring to FIGS. 6 and 7, as a first example, a plurality of main peaks may be formed in the light distribution curve. The light emitting device 100 may generate light so as to have a peak at an angle greater or less than a radiant intensity distribution angle of 0°. Through this, a maximum illuminance may be obtained even in a region far from the first target region S1. In particular, the second target region S2, which is far from the sterilization module 20, may have a uniform illuminance. Furthermore, the second target region S2 and the first target region S1 may have a uniform illuminance, which may be obtained on both the left and right sides based on the first target region (S2). More particularly, the sterilization module 20 may provide a uniform sterilization power to the sterilization region S.

Meanwhile, in the light emitting device 100, a peak at an angle smaller than 0 and a peak at an angle greater than 0 with respect to a radiant intensity distribution angle of 0 degrees may have different illuminances. An illuminance difference between these two peaks may be less than 20%.

Furthermore, the light emitting device 100 may generate light such that a sub-peak is formed at a radiant intensity distribution angle of 20° or more. Through this, the illuminance in a region relatively less distant from the center of the sterilization region S may be compensated. More particularly, the sterilization module 20 may provide a uniform sterilization power to the sterilization region S.

The light distribution curve of light that forms a radiation angle in the first virtual plane, among the light beams from the light emitting device 100 including the optical element 140 of the first example, is referred to as a first type light distribution curve. Furthermore, the light distribution curve of light that forms a radiation angle in the second virtual plane, among the light beams from the light emitting device 100 including the optical element 140 of the first example, is referred to as a second type light distribution curve.

The first type light distribution curve may have the largest intensity at a radiant intensity distribution angle of −20° or more and 20° or less. The plurality of main peaks of the first type light distribution curve may be formed with different intensities. Furthermore, one of the plurality of main peaks of the first type light distribution curve may have the largest intensity in the light distribution curve. The plurality of main peaks of the first type light distribution curve may include a first main peak P1 and a second main peak P2. The first main peak P1 and the second main peak P2 may be formed at a radiant intensity distribution angle of −20° or more and 20° or less.

The first main peak P1 may be formed at an angle smaller than a radiant intensity distribution angle of 0°. In particular, the first main peak P1 may be located at a radiant intensity distribution angle of −20° or more and 0° or less. The intensities of the first main peak P1 and the second main peak P2 may be formed differently. In particular, the intensity of the first main peak P1 may be formed to be smaller than the intensity of the second main peak P2. The normalized intensity of the first main peak P1 may be 0.8 or more and 1 or less. The first main peak P1 may also be positioned farther from the 0°radiant intensity distribution angle compared to the second main peak P2. A normalized intensity difference between the first main peak P1 and the second main peak P2 may be less than 0.2. This enables a uniform sterilization effect in the irradiation regions corresponding to the first main peak P1 and the second main peak P2.

The second main peak P2 may be formed at an angle greater than 0° radiant intensity distribution angle. In particular, the second main peak P2 may be located at a radiant intensity distribution angle of 0° or more and 20° or less. Furthermore, the intensity of the second main peak P2 may be formed to be greater than the intensity of the first main peak P1. The normalized intensity of the second main peak P2 may be 0.8 or more and 1 or less. This allows for a uniform sterilization effect according to the shape of the structure.

In the first type light distribution curve, a region between the first main peak P1 and the second main peak P2 may be formed to be concave. Hereinafter, the region between the first main peak P1 and the second main peak P2 in the first type light distribution curve is referred to as a first valley region. The normalized intensity of the first valley region may be 0.8 or more. In particular, the lowest intensity in the region between the first main peak P1 and the second main peak P2 may be 0.8 or more with respect to a higher intensity of the first main peak P1 and the second main peak P2. Such a first valley region may be formed such that an intensity decreases from the first main peak P1 to a predetermined reference angle, and the intensity increases from the reference angle to the second main peak P2. More particularly, the first valley region may have the smallest intensity at the reference angle. The reference angle may be located at −10° or more and −5° or less in the first type light distribution curve. Through this, it is possible to lower the normalized intensity of the center region, which is relatively close to the sterilization target, and to obtain a desired sterilization power with less power by efficiently distributing light.

Furthermore, a plurality of sub-peaks may be formed in the first type light distribution curve. The plurality of sub-peaks may have a smaller intensity than the plurality of main peaks. The plurality of sub-peaks may be located at −20° or less and 20° or more in the first type light distribution curve. Here, the sub-peak may have the highest intensity in a region where the intensity increases as the absolute value of the angle increases from the plurality of main peaks in the first type light distribution curve, and in a region where the intensity adjacent to the side where the absolute value of the angle increases decreases. The normalized intensity of the plurality of sub-peaks may be 0.4 or more with respect to a higher intensity of the first main peak P1 and the second main peak P2. By such a plurality of sub-peaks, the sterilization region S may be uniformly sterilized. The plurality of sub-peaks may include a first sub-peak SP1 and a second sub-peak SP2.

The first sub-peak SP1 may be formed at −30° or more and −20° or less. The normalized intensity of the first sub-peak SP1 may be 0.4 or more and 0.5 or less. A separation distance between the first sub-peak SP1 and the second sub-peak SP2 may be formed to be larger than a separation distance between the first main peak P1 and the second main peak P2. Through this, a uniform sterilization effect is provided up to the outer periphery of the sterilization region, thereby widening the design tolerance range for the outer periphery region and effectively lowering the design difficulty.

The second sub-peak SP2 may be formed at an angle of 20° or more and 30° or less. The normalized intensity of the second sub-peak SP2 may be 0.4 or more and 0.5 or less. The intensities of the second sub-peak SP2 and the first sub-peak SP1 may be formed differently, but it is not limited thereto, and they may be formed identically in other embodiments. A normalized intensity difference between the second sub-peak SP2 and the first sub-peak SP1 may be 0.1 or less. Through this, a uniform sterilization effect is provided, thereby widening the design tolerance range for the outer periphery region and effectively lowering the design difficulty.

Referring to FIG. 7, the second type light distribution curve may be formed similarly to the first type light distribution curve. In particular, a width of the second type light distribution curve and a width of the first type light distribution curve may be similar. Furthermore, a plurality of main peaks may be formed in the second type light distribution curve.

The second type light distribution curve may have the largest intensity at a radiant intensity distribution angle of −20° or more and 20° or less. The plurality of main peaks of the second type light distribution curve may be formed with different intensities. The plurality of main peaks of the second type light distribution curve may include a third main peak P3 and a fourth main peak P4. The third main peak P3 and the fourth main peak P4 may be formed at a radiant intensity distribution angle of −20° or more and 20° or less.

The third main peak P3 may be formed at an angle less than a 0° radiant intensity distribution angle. In particular, the third main peak P3 may be located at a radiant intensity distribution angle of −20° or more and 0° or less. The intensities of the third main peak P3 and the fourth main peak P4 may be formed differently. The intensity of the third main peak P3 may be formed to be greater than the intensity of the fourth main peak P4. The normalized intensity of the third main peak P3 may be 0.8 or more and 1 or less. The third main peak P3 may be formed at a position more spaced apart from the 0° radiant intensity distribution angle than the fourth main peak P4. An intensity difference between the third main peak P3 and the fourth main peak P4 may be less than 0.2.

The fourth main peak P4 may be formed at an angle greater than a 0° radiant intensity distribution angle. In particular, the fourth main peak P4 may be located at a radiant intensity distribution angle of 0° or more and 20° or less. Furthermore, the intensity of the fourth main peak P4 may be formed to be smaller than the intensity of the third main peak P3. The normalized intensity of the fourth main peak P4 may be 0.8 or more and 1 or less.

A difference in normalized intensity between the third main peak P3 and the fourth main peak P4 may be less than 0.2. Through this, a uniform sterilization effect may be obtained for each irrigation region.

In the second type light distribution curve, a region between the third main peak P3 and the fourth main peak P4 may be formed to be concave. Hereinafter, the region between the third main peak P3 and the fourth main peak P4 in the second type light distribution curve is referred to as a second valley region. The intensity of the second valley region may be 0.8 or more. In particular, the lowest intensity in the region between the third main peak P3 and the fourth main peak P4 may be 0.8 or more with respect to a higher intensity of the third main peak P3 and the fourth main peak P4. The second valley region may be formed such that an intensity decreases from the third main peak P3 to a predetermined reference angle, and the intensity increases from the reference angle to the fourth main peak P4. In particular, the second valley region may have the smallest intensity at the reference angle. The reference angle may be located at −10° or more and 10° or less in the second type light distribution curve. Through this, it is possible to lower the normalized intensity of the center region, which is relatively close to the sterilization target, and to obtain a desired sterilization power with less power by efficiently distributing light.

Furthermore, a plurality of sub-peaks may be formed in the second type light distribution curve. The plurality of sub-peaks of the second type light distribution curve may have a smaller intensity than the plurality of main peaks of the second type light distribution curve. The plurality of sub-peaks of the second type light distribution curve may be located at −20° or less and 20° or more in the second type light distribution curve. Here, the plurality of sub-peaks may have the highest intensity in a region where the intensity increases as the absolute value of the angle increases from the plurality of main peaks in the second type light distribution curve, and in a region where the intensity adjacent to the side where the absolute value of the angle increases decreases. The intensity of the plurality of sub-peaks may be 0.4 or more with respect to a higher intensity of the third main peak P3 and the fourth main peak P4. By such a plurality of sub-peaks, the sterilization region S may be uniformly sterilized. The plurality of sub-peaks of the second type light distribution curve may include a third sub-peak SP3 and a fourth sub-peak SP4.

The third sub-peak SP3 may be formed at −30° or more and −20° or less. The normalized intensity of the third sub-peak SP3 may be 0.4 or more and 0.5 or less. A separation distance between the third sub-peak SP3 and the fourth sub-peak SP4 may be formed to be larger than a separation distance between the third main peak P3 and the fourth main peak P4. Through this, a uniform sterilization effect is provided up to the outer periphery of the sterilization region, thereby widening the design tolerance range for the outer periphery region and effectively lowering the design difficulty.

The fourth sub-peak SP4 may be formed at 20° or more and 30 or less. The normalized intensity of the fourth sub-peak SP4 may be 0.4 or more and 0.5 or less. The intensities of the fourth sub-peak SP4 and the third sub-peak SP3 may be formed differently, but it is not limited thereto, and they may be formed identically in other embodiments. A normalized intensity difference between the fourth sub-peak SP4 and the third sub-peak SP3 may be less than 0.1. Through this, a uniform sterilization effect is provided up to the outer periphery of the sterilization region, thereby widening the design tolerance range for the outer periphery region and effectively lowering the design difficulty.

To implement the first type light distribution curve or the second type light distribution curve, a height of the optical element 140 may be twice or more the long axis of the light-emitting surface of the light emitter 130. More specifically, it may be 3 times or more and 4 times or less. Alternatively, a bottom region of the optical element 140 may be 20 times or more that of the light emitter 130. Still alternatively, a radius of curvature in a region corresponding to the light-emitting surface of the light emitter 130at the center of the optical element 140 may be longer than a length of one axis of the light-emitting surface. More specifically, it may be 2 times or more. In addition, a center of the radius of curvature of the optical element 140 may be disposed in a region higher than a bottom surface of the optical element 140. For example, a height from the bottom surface to the highest point of the optical element 140 may be higher than a radius of the bottom of the optical element 140.

A difference between the first type light distribution curve and the second type light distribution curve may vary depending on the direction of the light emitter 130. The first type light distribution curve may be obtained by measuring a radiation angle in a direction from a first electrode to a second electrode of the light emitter 130. The second type light distribution curve may be obtained by measuring a radiation angle in a plane direction perpendicular to the direction in which the first electrode and the second electrode of the light emitter are disposed.

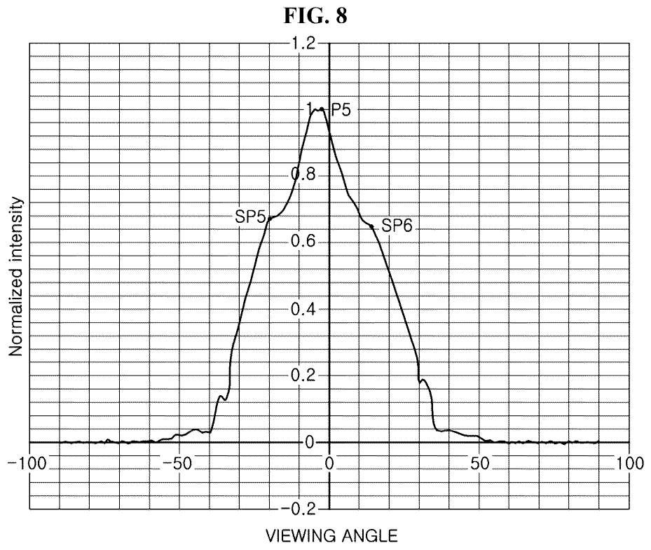

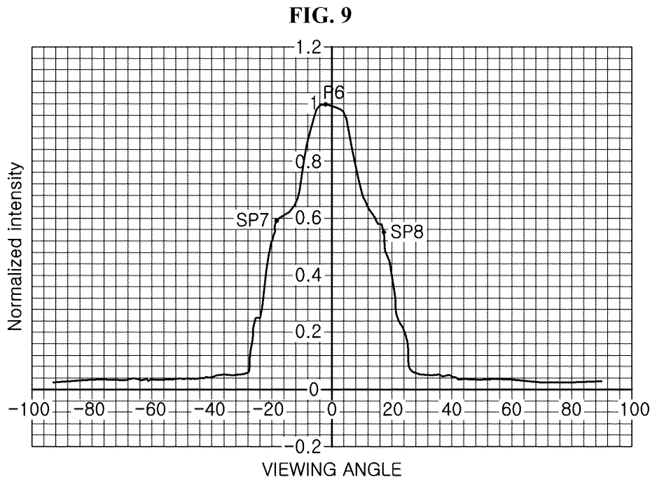

Referring to FIGS. 8 and 9, as a second example, the light emitting device 100 may generate light in which a single main peak is formed in the light distribution curve. The light emitting device 100 may have a maximum normalized luminous intensity (main peak) at an angle of −10° or more to less than 10° with respect to a radiant intensity distribution angle of 0°. The light emitting device 100 may generate light having a narrow orientation angle. The light emitting device 100 may increase the sterilization power by concentrating high-intensity light on a relatively narrow sterilization region S through a narrow orientation angle.

Furthermore, the light emitting device 100 may generate light that may have a sub-peak at an angle of −20° to 20°. The sub-peak may have a lower luminous intensity than the maximum normalized luminous intensity. The luminous intensity at the sub-peak may be 60% or more and less than 80% of the maximum peak. The light emitting device 100 may provide sufficient sterilization power even to the second target region S2 spaced apart from the first target region S1 through the sub-peak.

The light distribution curve of light that forms a radiation angle in a first virtual plane, among the light emitted from the light emitting device 100 including the optical element 140 of such a second example, is referred to as a third type light distribution curve. Furthermore, the light distribution curve of the light that forms a radiation angle in a second virtual plane, among the light emitted from the light emitting device 100 including the optical element 140 of the second example, is referred to as a fourth type light distribution curve.

A single fifth main peak P5 may be formed in the third type light distribution curve. The third type light distribution curve may have the largest intensity at a radiant intensity distribution angle of −10° or more and less than 10°. In particular, the fifth main peak P5 may be formed at a radiant intensity distribution angle of −10° or more and 10° or less. Due to the characteristics of the third type light distribution curve, light emitted from the optical element 140 may have a narrow radiation angle, so that a small region may be intensively sterilized. More particularly, when the sterilization region S is formed small, light emitted from the optical element 140 may intensively sterilize the small sterilization region S. The normalized intensity of the fifth main peak P5 may be 0.9 or more and 1 or less. For the fifth main peak P5 , a region that maintains a normalized intensity of 0.9 to 1 may extend over an angular width of 5 degrees or more. Through this, the narrow sterilization region S may be uniformly irradiated with light.

Furthermore, a plurality of sub-peaks smaller than the fifth main peak P5 may be formed in the third type light distribution curve. The plurality of sub-peaks may be formed at −30° or less and 30° or more. Furthermore, the plurality of sub-peaks may have the highest intensity in a region where the intensity increases as the absolute value of the angle increases from the fifth main peak P5, and in a region where the intensity adjacent to the side where the absolute value of the angle increases decreases. Furthermore, the intensity of the plurality of sub-peaks may be 0.6 or more and 0.8 or less. By the plurality of sub-peaks, the sterilization region S may be uniformly sterilized. The plurality of sub-peaks may include a fifth sub-peak SP5 and a sixth sub-peak SP6.

The fifth sub-peak SP5 may be formed at a radiant intensity distribution angle of −25° or more and −10° or less. The intensities of the fifth sub-peak SP5 and the sixth sub-peak SP6 may be formed differently. In particular, the intensity of the fifth sub-peak SP5 may be formed to be greater than the intensity of the sixth sub-peak SP6. A normalized intensity difference between the intensity of the fifth sub-peak SP5 and the sixth sub-peak SP6 may be 0.1 or less. Through this, the design tolerance range can be widened and the design difficulty can be effectively lowered.

The sixth sub-peak SP6 may be formed at a radiant intensity distribution angle of 10° or more and 25° or less. The intensity of the sixth sub-peak SP6 may be formed to be smaller than the intensity of the fifth sub-peak SP5. Furthermore, the sixth sub-peak SP6 may be located closer to 0° than the fifth sub-peak SP5.

Referring to FIG. 9, a single sixth main peak P6 may be formed in the fourth type light distribution curve. The fourth type light distribution curve may be formed similarly to the third type light distribution curve. A width of the fourth type light distribution curve may be similar to a width of the third type light distribution curve. The third type light distribution curve may have the largest intensity at a radiant intensity distribution angle of −10° or more and less than 10°. In particular, the sixth main peak P6 may be formed at a radiant intensity distribution angle of −10° or more and 10° or less. The intensity of the sixth main peak P6 may be the same as the intensity of the fifth main peak P5. The normalized intensity of the sixth main peak P6 may be 0.9 or more and 1 or less. Due to the characteristics of the fourth type light distribution curve, light emitted from the optical element 140 may have a narrow radiation angle, so that a small region may be intensively sterilized. In particular, when the sterilization region S is formed small, light emitted from the optical element 140 may intensively sterilize the small sterilization region S. For the sixth main peak P6, a region that maintains a normalized intensity of 0.9 to 1 may extend over an angular width of 10 degrees or more. Through this, the narrow sterilization region S may be uniformly irradiated with light.

Furthermore, a plurality of sub-peaks smaller than the sixth main peak P6 may be formed in the third type light distribution curve. The plurality of sub-peaks may be formed at −10° or less and 10° or more. Furthermore, the plurality of sub-peaks may have the highest intensity in a region where the intensity increases as the absolute value of the angle increases from the sixth main peak P6, and in a region where the intensity adjacent to the side where the absolute value of the angle increases decreases. Furthermore, the normalized intensity of the plurality of sub-peaks may be 0.5 or more and 0.7 or less. By such the plurality of sub-peaks, the sterilization region S may be uniformly sterilized. The plurality of sub-peaks may include a seventh sub-peak SP7 and an eighth sub-peak SP8.

The seventh sub-peak SP7 may be formed at a radiant intensity distribution angle of −25° or more and −15° or less. The intensities of the seventh sub-peak SP7 and the eighth sub-peak SP8 may be formed differently. In particular, the intensity of the seventh sub-peak SP7 may be formed to be greater than the intensity of the eighth sub-peak SP8.

The eighth sub-peak SP8 may be formed at a radiant intensity distribution angle of 15° or more and 25° or less. The intensity of the eighth sub-peak SP8 may be formed to be smaller than the intensity of the seventh sub-peak SP7. Furthermore, the eighth sub-peak SP8 may be located closer to 0° than the seventh sub-peak SP7.

To implement the third type light distribution curve or the fourth type light distribution curve, a height of the optical element 140 may be 2 times or more and less than 3 times the long axis of the light-emitting surface of the light emitter 130. When the light emitter 130 is a rectangle, the height may be 1.5 times or more and less than 2 times the short axis. Alternatively, a bottom region of the optical element 140 may be 9 times or more that of the light emitter 130. Still alternatively, a radius of curvature in a region corresponding to the light-emitting surface of the light emitter 130 at the center of the optical element 140 may be shorter than a length of one long axis of the light-emitting surface. In addition, a center of the radius of curvature of the optical element 140 may be disposed in a region higher than a bottom surface of the optical element 140. For example, a height from the bottom surface to the highest point of the optical element 140 may be higher than a radius of the bottom of the optical element 140.

A difference between the third type light distribution curve and the fourth type light distribution curve may vary depending on the direction of the light emitter 130. The third type light distribution curve may be obtained by measuring a radiation angle in a direction from a first electrode to a second electrode of the light emitter 130. The third type light distribution curve may be obtained by measuring a radiation angle in the long axis direction of the light emitter 130. The fourth type light distribution curve may be obtained by measuring a radiation angle in a plane direction perpendicular to the direction in which the first electrode and the second electrode of the light emitter are disposed. The fourth type light distribution curve may be obtained by measuring a radiation angle in the direction in which the short axis of the light emitter is disposed.

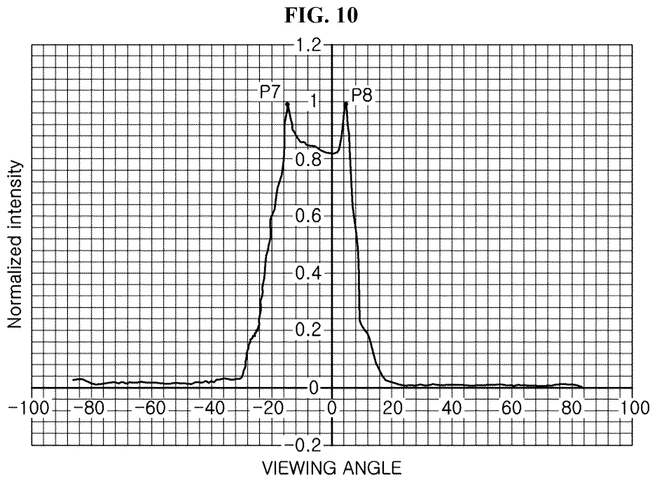

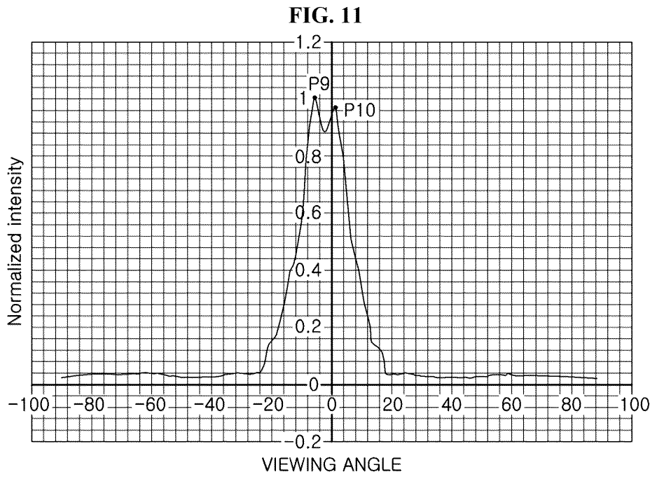

Referring to FIGS. 10 and 11, as a third example, the optical element 140 may refract light such that a plurality of main peaks are formed in the light distribution curve. The intensities of the plurality of main peaks may be formed to be the same or different from each other. The light distribution curve of the light that forms a radiation angle in a first virtual plane, among the light emitted from the light emitting device 100 including the optical element 140 of the third example, is referred to as a fifth type light distribution curve. Furthermore, the light distribution curve of the light that forms a radiation angle in a second virtual plane, among the light emitted from the light emitting device 100 including the optical element 140 of a fourth example, is referred to as a sixth type light distribution curve.

Furthermore, the fifth type light distribution curve and the sixth type light distribution curve may be formed differently. In particular, a separation distance between the plurality of main peaks in the fifth type light distribution curve may be formed to be larger than a separation distance between the plurality of main peaks in the sixth type light distribution curve. More particularly, a radiation angle of the light distributed in the long axis direction among the light of the light emitting device 100 of the third example may be formed to be larger than a radiation angle of the light distributed in the short axis direction.

The fifth type light distribution curve may have the largest intensity at a radiant intensity distribution angle of −30° or more and 10° or less. The intensities of the plurality of main peaks of the fifth type light distribution curve may be formed to be different or the same. The plurality of main peaks of the fifth type light distribution curve may include a seventh main peak P7 and an eighth main peak P8. The seventh main peak P7 and the eighth main peak P8 may be formed at −30° or more and 10° or less in the fifth type light distribution curve.

The seventh main peak P7 may be formed at an angle less than a 0° radiant intensity distribution angle. In particular, the seventh main peak P7 may be located at −30° or more and 20° or less. The intensities of the seventh main peak P7 and the eighth main peak P8 may be formed differently. In particular, the intensity of the seventh main peak P7 may be formed to be smaller than the intensity of the eighth main peak P8. The normalized intensity of the seventh main peak P7 may be 0.8 or more and 1 or less. The seventh main peak P7 may be disposed at a position more spaced apart from the 0° radiant intensity distribution angle than the eighth main peak P8. An intensity difference between the seventh main peak P7 and the eighth main peak P8 may be less than 0.2. Furthermore, a normalized intensity difference between the seventh main peak P7 and the eighth main peak P8 may be less than 0.1. Through this, the design tolerance range can be widened and the design difficulty can be effectively lowered.

The eighth main peak P8 may be formed at an angle greater than a 0° radiant intensity distribution angle. In particular, the eighth main peak P8 may be located at a radiant intensity distribution angle of 0° or more and 10° or less. Furthermore, the intensity of the eighth main peak P8 may be formed to be greater than the intensity of the seventh main peak P7. The normalized intensity of the eighth main peak P8 may be 0.9 or more and 1 or less.

Furthermore, in the fifth type light distribution curve, a region between the seventh main peak P7 and the eighth main peak P8 may be formed to be concave. Hereinafter, the region between the seventh main peak P7 and the eighth main peak P8 in the fifth type light distribution curve is referred to as a third valley region. The normalized intensity of the third valley region may be 0.8 or more. In particular, the lowest intensity in the region between the seventh main peak P7 and the eighth main peak P8 may be 0.8 or more with respect to a higher intensity of the seventh main peak P7 and the eighth main peak P8. The third valley region may be formed such that an intensity decreases from the seventh main peak P7 to a predetermined reference angle, and the intensity increases from the predetermined reference angle to the eighth main peak P8. In particular, the third valley region may have the smallest intensity at the predetermined reference angle. The predetermined reference angle of the third valley region may be located at −5° or more and 5° or less in the fifth type light distribution curve. Furthermore, a width of the third valley region may be formed to be larger than a width of a fourth valley region to be described later.

Referring to FIG. 11, the sixth type light distribution curve may have the largest intensity at a radiant intensity distribution angle ranging from −10° to 5°. The intensities of the plurality of main peaks of the sixth type light distribution curve may be formed to be different or the same. The plurality of main peaks of the sixth type light distribution curve may include a ninth main peak P9 and a tenth main peak P10.

The ninth main peak P9 may be formed at a radiant intensity distribution angle smaller than 0°. In particular, the ninth main peak P9 may be located at a radiant intensity distribution angle ranging from −10° to 0°. The intensities of the ninth main peak P9 and the tenth main peak P10 may be formed differently. In particular, the intensity of the ninth main peak P9 may be formed to be greater than the intensity of the tenth main peak P10. The normalized intensity of the ninth main peak P9 may be 0.9 or more and 1 or less. A normalized intensity difference between the ninth main peak P9 and the tenth main peak P10 may be less than 0.1. Through this, the design tolerance range can be widened and the design difficulty can be effectively lowered. The ninth main peak P9 may be disposed at a position more spaced apart from the radiant intensity distribution angle of 0° than the tenth main peak P10. An intensity difference between the ninth main peak P9 and the tenth main peak P10 may be less than 0.2. Through this, the narrow sterilization region S may be uniformly sterilized.

The tenth main peak P10 may be formed at an angle greater than the radiant intensity distribution angle of 0°. In particular, the tenth main peak P10 may be located at a radiant intensity distribution angle of 0° or more and 5° or less. Furthermore, the intensity of the tenth main peak P10 may be formed to be smaller than the intensity of the ninth main peak P9. The normalized intensity of the tenth main peak P10 may be 0.8 or more and 1 or less.

Furthermore, in the sixth type light distribution curve, a region between the ninth main peak P9 and the tenth main peak P10 may be formed to be concave. Hereinafter, the region between the ninth main peak P9 and the tenth main peak P10 in the sixth type light distribution curve is referred to as a fourth valley region. The normalized intensity of the fourth valley region may be 0.8 or more. In particular, the lowest intensity in the region between the ninth main peak P9 and the tenth main peak P10 may be 0.8 or more with respect to a higher intensity of the ninth main peak P9 and the tenth main peak P10. The fourth valley region may be formed such that an intensity decreases from the ninth main peak P9 to a predetermined reference angle, and the intensity increases from the reference angle to the tenth main peak P10. In particular, the fourth valley region may have the smallest intensity at the predetermined reference angle. The predetermined reference angle of the fourth valley region may be located at a radiant intensity distribution angle ranging from −5° to 0° in the sixth type light distribution curve. Furthermore, a width of the fourth valley region may be formed to be smaller than the width of the third valley region. Through this, the design tolerance range can be widened and the design difficulty can be effectively lowered.

To implement the fifth type light distribution curve or the sixth type light distribution curve, a height of the optical element 140 may be 2 times or more and less than 3 times the long axis of the light-emitting surface of the light emitter 130. When the light emitter 130 is a rectangle, the height may be 2 times or more and less than 4 times the short axis. Alternatively, a bottom region of the optical element 140 may be 10 times or more that of the light emitter 130. Still alternatively, a radius of curvature in a region corresponding to the light-emitting surface of the light emitter 130 at the center of the optical element 140 may be shorter than a length of one long axis of the light-emitting surface. In addition, a center of the radius of curvature of the optical element 140 may be disposed in a region higher than a bottom surface of the optical element 140. As a method of implementing this, a height from the bottom surface to the highest point of the optical element 140 may be higher than a radius of the bottom of the optical element 140.

A difference between the fifth type light distribution curve and the sixth type light distribution curve may vary depending on the direction of the light emitter 130. The fifth type light distribution curve may be obtained by measuring a radiation angle in a direction from a first electrode to a second electrode of the light emitter 130. The fifth type light distribution curve may be obtained by measuring a radiation angle in the long axis direction of the light emitter 130. The sixth type light distribution curve may be obtained by measuring a radiation angle in a plane direction perpendicular to the direction in which the first electrode and the second electrode of the light emitter are disposed. The sixth type light distribution curve may be obtained by measuring a radiation angle in the direction in which the short axis of the light emitter is disposed.

Meanwhile, the first light emitting device of the first sterilization module 20a may emit light having a first light distribution curve for sterilizing the first region Sa. The first light distribution curve may include the fifth type light distribution curve and the sixth type light distribution curve, but is not limited thereto, and may include the first type light distribution curve and the second type light distribution curve. In particular, the first light emitting device of the first sterilization module 20a may generate light for forming the fifth type light distribution curve and the sixth type light distribution curve. Such a first light emitting device may emit light having a wide radiation angle in the vertical direction corresponding to the shape of the tank 12, and emit light having a narrow radiation angle in the horizontal direction. Accordingly, the first light emitting device may efficiently sterilize the tank 12 which has a long length in the vertical direction and a narrow length in the horizontal direction. In the first light distribution curve, a plurality of main peaks may be formed at a radiant intensity distribution angle ranging from −30° to 20°. Furthermore, an intensity of the first light distribution curve at a radiant intensity distribution angle ranging from −30° to 20° may be 0.8 or more with respect to a higher intensity of the plurality of main peaks.

The second light emitting device of the second sterilization module 20b may generate light having a second light distribution curve for sterilizing the second region Sb. The second light distribution curve may include the third type light distribution curve and the fourth type light distribution curve. In particular, a single main peak may be formed in the second light distribution curve. The main peak of the second light distribution curve may be formed at a radiant intensity distribution angle ranging from −10° to 10°.

Hereinafter, the operation and effects of the light emitting device 100 and the sterilizer 1 including the same according to the first embodiment of the invention will be described.

According to the first embodiment of the invention, the light emitting device 100 may sterilize the sterilization region S. In particular, the first sterilization module 20a may generate light to sterilize the tank 12. The second sterilization module 20b may generate light to sterilize the flow channel 14.

The light emitting device 100 may have a clear color reproduction rate.

Furthermore, since the light emitting device 100 may protect the light emitter 130 from the external environment, reliability may be increased.

Furthermore, the light emitting device 100 may be formed as a high-quality light emitting device with reduced chromatic aberration.

Furthermore, the light emitting device 100 may efficiently sterilize the first region Sa in which the length in the vertical direction and the length in the horizontal direction are formed differently.

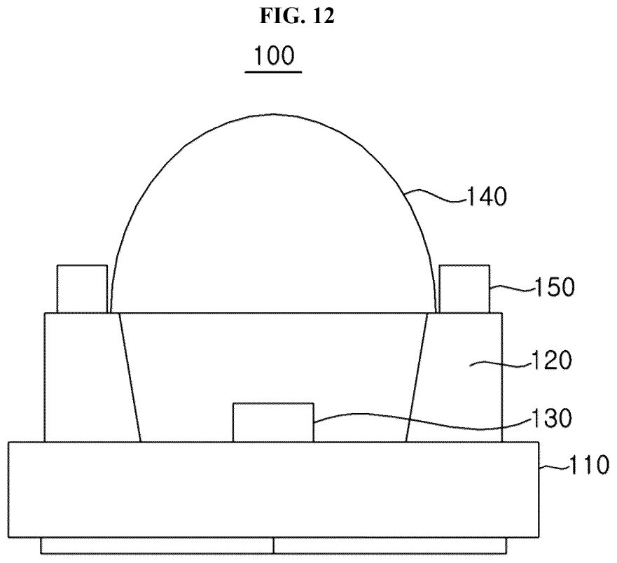

Hereinafter, a light emitting device 100 and a sterilizer 1 including the same according to a second embodiment of the invention will be described with reference to FIG. 12. In describing the second embodiment, the light emitting device 100 further includes a sidewall 150 as compared to the first embodiment, and thus this difference will be mainly described.

The sidewall 150 may extend upward from an upper side of the housing 120. An optical element 140 may be disposed inside such a sidewall 150. The sidewall 150 may reflect light emitted from the optical element 140 upward. A height of the sidewall 150 may be smaller than a height of the optical element 140. Furthermore, due to the sidewall 150, an edge of the optical element 140 may be disposed closer to an inner surface than an outer surface of the housing 120. Such a sidewall 150 may protect the optical element 140 from external impact.

The optical element 140 of the second embodiment may have a smaller cross-sectional region than an outer peripheral surface of the housing 120. Furthermore, the optical element 140 may have the smallest radius of curvature in a region that overlaps perpendicularly with the light-emitting surface of the light emitter 130. Through this, a light emitting device having third to fifth type light distribution curves may be implemented.

Hereinafter, the operation and effects of the light emitting device 100 and the sterilizer 1 including the same according to the second embodiment of the invention will be described.

Since the sidewall 150 may reflect the light emitted from the optical element 140 upward, light extraction efficiency may be increased.

Furthermore, the sidewall 150 may protect the optical element 140.

Light emitting devices constructed according to embodiments of the invention are capable of uniformly sterilizing a sterilization region.

Light emitting devices according to embodiments of the invention are also capable of efficiently emitting light by increasing the light extraction efficiency.

Light emitting devices according to embodiments of the invention are also capable of having a clear color reproduction rate.

Light emitting devices according to embodiments of the invention are also capable of increasing reliability by protecting a light-emitting element from an external environment.

Light emitting devices according to embodiments of the invention are also capable of implementing a high-quality light emitting device with reduced chromatic aberration.

Light emitting devices according to embodiments of the invention are also capable of improving light collection efficiency by concentrating light from a light source unit onto a light irradiation region.

Light emitting devices according to embodiments of the invention may provide a light emitting device that efficiently emits light by increasing light extraction efficiency.

Light emitting devices according to embodiments of the invention may also provide a light emitting device with improved reliability by efficiently dissipating heat to increase heat dissipation efficiency.

Furthermore, exemplary embodiments of the disclosed technology may provide a light emitting device with improved luminance by adjusting the refraction direction.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A light emitting device, comprising:
a device substrate;
a device housing disposed on the device substrate and including a cavity that exposes at least a region of the device substrate to an outside;
a light emitter disposed on the device substrate and having a light-emitting surface configured to emit light; and
an optical element disposed above the device housing and configured to refract light emitted from the light emitter,
wherein the optical element includes a surface including:
a first region that intersects a first virtual line extending from a center of the light emitter at a first angle with respect to the light-emitting surface and having a first curvature; and
a second region that intersects a second virtual line extending from the center of the light emitter at a second angle different from the first angle with respect to the light-emitting surface and having a second curvature different from the first curvature.

2. The light emitting device of claim 1, wherein a center region of the surface of the optical element overlaps the light emitter and has a largest curvature among regions of the surface.

3. The light emitting device of claim 2, wherein a center of curvature of the center region is disposed above the light emitter.

4. The light emitting device of claim 1, wherein an edge region of the surface of the optical element is adjacent to the device housing and has the smallest curvature.

5. The light emitting device of claim 1, wherein the optical element is configured to refract light to form a light distribution curve with an illuminance of 50% or more within a radiant intensity distribution angle ranging from −45° to 45°.

6. The light emitting device of claim 5, wherein the light distribution curve includes a plurality of main peaks.

7. The light emitting device of claim 6, wherein the plurality of main peaks comprises:
a first main peak formed at a radiant intensity distribution angle less than 0°; and
a second main peak formed at a radiant intensity distribution angle greater than 0°.

8. The light emitting device of claim 7, wherein normalized intensities of the first main peak and the second main peak are different from each other.

9. The light emitting device of claim 7, wherein a lowest normalized intensity in a region between the first main peak and the second main peak is at least 0.8 times the higher of the normalized intensities of the first main peak and the second main peak.

10. The light emitting device of claim 7, wherein the first main peak and the second main peak are formed at the radiant intensity distribution angle ranging from −20° to 20° in the light distribution curve.

11. The light emitting device of claim 5, wherein the light distribution curve includes a single main peak within the radiant intensity distribution angle ranging from −20° to 20°.

12. The light emitting device of claim 6, wherein the light distribution curve includes a plurality of sub-peaks, the plurality of sub-peaks having a smaller normalized intensity than the plurality of main peaks and being formed at a larger radiant intensity distribution angle than the main peaks, and
wherein the plurality of sub-peaks are formed at the radiant intensity distribution angle ranging from −20° to 20°.

13. The light emitting device of claim 12, wherein normalized intensities of the plurality of sub-peaks are at least 0.4 times the higher of the normalized intensities of the plurality of the main peaks.

14. A light emitting device, comprising:
a substrate;
a housing disposed on the substrate and including a cavity exposing at least a region of the substrate to an outside;
a light emitter disposed in the region of the substrate and configured to generate light;
an optical element supported on an upper side of the housing and configured to refract light emitted from the light emitter; and
a plurality of sidewalls extending upward from an upper side of the housing,
wherein the optical element is disposed between the plurality of sidewalls.

15. A sterilizer, comprising:
a housing assembly that provides a first region and a second region;
a first light emitting device configured to generate light having a first light distribution curve to sterilize the first region; and
a second light emitting device configured to generate light having a second light distribution curve to sterilize the second region,
wherein the first light distribution curve and the second light distribution curve have main peaks at different radiant intensity distribution angles.

16. The sterilizer of claim 15, wherein the first region is larger than the second region.

17. The sterilizer of claim 15, wherein the first light emitting device provides a wider radiation angle than the second light emitting device.

18. The sterilizer of claim 15, wherein the first light distribution curve includes a plurality of main peaks, and
wherein the plurality of main peaks are formed within the radiant intensity distribution angle ranging from −30° to 20°.

19. The sterilizer of claim 15, wherein a normalized intensity of the first light distribution curve at the radiant intensity distribution angle of −30° to 20° is at least 0.8 times the higher of normalized intensities of the plurality of the main peaks.

20. The sterilizer of claim 15, wherein the second light distribution curve includes a single main peak.
